(19) United States
(12) Patent Application Publication
(10) Pub. No.: US 2024/0166987 A1
(43) Pub. Date: May 23, 2024

(54) BIOELECTRODE FOR INCREASING ABUNDANCE OF CABLE BACTERIA AND USING METHOD THEREOF

(71) Applicant: SHENZHEN POLYTECHNIC UNIVERSITY, SHENZHEN (CN)

(72) Inventors: HUAN WANG, SHENZHEN (CN); YANG ZHOU, SHENZHEN (CN); LIJUN ZHU, SHENZHEN (CN); SHAOFENG LI, SHENZHEN (CN); LIUYI CHEN, SHENZHEN (CN); CANLIANG HUANG, SHENZHEN (CN); QIANWEN ZHAO, SHENZHEN (CN); CHENGWEI YAN, SHENZHEN (CN)

(21) Appl. No.: 18/475,194

(22) Filed: Sep. 26, 2023

Related U.S. Application Data
(63) Continuation of application No. PCT/CN2022/139104, filed on Dec. 14, 2022.

(30) Foreign Application Priority Data
Nov. 18, 2022 (CN) 202211447284.X

(51) Int. Cl. C12N 1/20; C12N 13/00; C12Q 1/04; C12Q 1/6869

(52) U.S. Cl. CPC C12N 1/20; C12N 13/00; C12Q 1/04; C12Q 1/6869

(57) ABSTRACT

The present invention relates to a bioelectrode for increasing abundance of cable bacteria, including an anode, a cathode, and a partition. The anode is horizontally disposed, and the cathode is disposed perpendicular to the anode. The anode and the cathode are separated by using the partition. An objective of the present invention is to provide a method for keeping a high abundance of the cable bacteria in sediments when oxygen content of overlying water is low and even in an anaerobic environment. The method can be further performed in a self-starting manner, and has a potential application prospect in ecological remediation of sediments.

### TECHNICAL FIELD

The present invention belongs to the field of sediment remediation, and in particular, relates to a bioelectrode for increasing an abundance of cable bacteria and a using method thereof.

### BACKGROUND

A sulfur oxidation process is an important part for Earth sulfur element cycling. In various sediments, sulfate is usually reduced to sulfide in an anaerobic environment, and microorganisms usually need to be in contact with a suitable electron acceptor to oxidize the sulfide to elemental sulfur or sulfate, etc., which form a sulfur cycle. However, the sulfide is usually formed in a lower layer of sediments, and an electron acceptor such as oxygen is located in a surface layer. This spatial separation requires a cycle of a sulfur element to be realized by long-distance electron transfer. Cable bacteria are long and thread-like multicellular microorganisms with a capability of “centimeter-scale long-distance” electron transfer. The cable bacteria are used to couple oxygen reduction on a surface of the sediments with sulfide oxidation in the deep sediments through electric current. This way of electrogenic sulfur oxidation makes the cable bacteria play a very important role in the sediment sulfur cycle. In addition, the electrogenic sulfur oxidation can significantly affect a cycle of sulfur, iron, carbon, calcium, nitrogen, phosphor, and other elements, relieve toxicity of the sulfide, and strengthen degradation of organic pollutants. Therefore, maintenance of high-abundance cable bacteria in the sediments is of great significance for long-term stability of an ecosystem and ecological remediation of polluted sediments.

Maintenance of population activity of cable bacteria usually needs to meet three conditions: dissolved oxygen content of the overlying water is greater than 50 μmol/L, sulfide content in the sediments is high, and bioturbation is small. Previous studies have shown that increasing dissolved oxygen content of overlying water through aeration can increase the abundance of the cable bacteria in the sediments and promote removal of various organic substances in the sediments. However, in the current technology, when cable bacteria are applied to sediment ecological remediation, instability of the dissolved oxygen content in the overlying water will make population abundance of the cable bacteria very low or even undetectable, and consequently, effect of the cable bacteria in sediment ecological remediation cannot be brought into play. The existing method for increase the abundance of the cable bacteria in the sediment is to maintain high dissolved oxygen content in the overlying water through aeration. However, it is usually difficult to achieve large-scale aeration in actual ecological remediation, and long-term aeration can lead to huge energy consumption and high maintenance costs. Therefore, it is extremely uneconomical to maintain the cable bacteria in an oxygen-rich environment for a long time.

Therefore, how to find a method to maintain a high abundance of the cable bacteria in the anaerobic sediments of the overlying water is obviously of great significance for application of the cable bacteria in ecological remediation.

### SUMMARY

Based on the disadvantages, an objective of the present invention is to provide a method for keeping a high abundance of cable bacteria in sediments when oxygen content of overlaying water is extremely low and even in an anaerobic environment. The method can be combined with bioelectrochemical remediation, and has a potential application prospect in ecological remediation of the sediments.

An objective of the present invention is to provide a bioelectrode for increasing an abundance of cable bacteria, including an anode, a cathode, and a partition;

The anode is horizontally disposed, and the cathode is disposed perpendicular to the anode;

The anode and the cathode are separated by using the partition.

Generally, the anode may be disposed above a horizontal base, and the base is inserted into sediments and fastened. The anode is just disposed on a surface of the sediments when used. A perpendicular fastening bar is disposed on an upper portion of the anode, and the cathode is disposed on the fastening bar, so that the anode and the cathode are in a state perpendicular to each other, without direct contact, and are separated by using the partition.

Advantages of this setting are that: A water level of the overlying water of the sediments is affected by different application sites, seasonal changes, and the like. The cathode is placed vertically in the overlying water, so that a portion of an electrode can always be in contact with a gas-liquid interface during application of the apparatus, to maintain an electron transfer path. In this way, the apparatus is not affected by a change of the water level of the overlying water during an application process, so that a scope of application of the apparatus is greatly increased and maintenance costs are reduced. The bioanode is parallel to a surface of the sediments, and can be in contact with the sediments in a large area, maintain the abundance of the cable bacteria in the sediments, and maximize a range of influence of a bioelectrode.

Further, the anode and/or the cathode are/is made of a carbonaceous material.

Preferably, the carbonaceous material may be a carbon brush or another carbonaceous material such as a carbon felt.

Further, the partition is made of an insulation material such as polyethylene, polypropylene, polyvinyl chloride, etc.

Further, the anode and/or the cathode are/is carbon brush electrodes/a carbon brush electrode.

Further, a resistor or a potentiostat is connected between the anode and the cathode.

Another objective of the present invention is to provide a using method of a bioelectrode for increasing an abundance of cable bacteria, including the following steps:
S1: inoculating cable bacteria into to-be-remediated sediments;
S2: keeping aeration lasting for 14 to 21 d in overlying water added in step S1 or original overlying water;
S3: inserting the bioelectrode for increasing the abundance of the cable bacteria into the sediments, keeping a constant potential of the anode, stopping the aeration, and sealing the entire system;
S4: maintaining for 5 to 60 d after the system reaches an anaerobic condition, taking the sediments again to test cable bacteria abundance in the sediments, and recording the cable bacteria abundance as D.

Further, the anaerobic condition is that oxygen content in the overlying water is less than or equal to 0.4 mg/L.

Further, the constant potential of the anode is −200 mV to 400 mV. The electrode potential range is conducive to transfer the electrons produced by microorganisms to the cathode from the anode, and finally to oxygen, which form a complete electronic path. Ethier too high or too low a potential will affect the abundance of the cable bacteria in the sediments due to the obstruction of the electronic path. The too low potential will prevent the electrons produced by the microorganisms from being transferred to the bioanode, and the too high anode potential is not conducive to the transfer of electrons generated by microorganisms to the cathode from the anode. This will eventually lead to reduction of the abundance of the cable bacteria in the sediments due to lack of electron acceptors.

Further, a method for testing the abundance of the cable bacteria in the sediments is a 16s RNA sequencing method.

The present invention has the following beneficial effects:
1. The technical solution of the present invention can maintain a high abundance of the cable bacteria in the sediments when the oxygen content of the overlying water is extremely low by controlling the anode potential. After the bioanode is successfully enriched, a potential range that can effectively maintain the abundance of the cable bacteria can be maintained without continuing to apply potential. This shows that the method features low energy consumption.
2. The technical solution in the present invention can accelerate the sulfur cycle process in the sediments. This is because, on one hand, due to the reduction of oxygen content in the overlying water, reduction of sulfate radicals and generation of sulfide in the surface sediments can be accelerated, and the generated sulfide can be further used for growth of the cable bacteria, to maintain the abundance of the cable bacteria. On the other hand, the acceleration of sulfur cycle can promote a carbon cycle in the sediments, so that the purpose of promoting the remediation of polluted sediments is achieved. This shows that this method has effect of strengthening remediation effect.
3. In the application field of in-situ remediation of the polluted sediments, this technical solution can be combined with bioelectrochemical remediation, and the bioanode can be self-started without additional energy input, to maintain the abundance of the cable bacteria in the sediments. This solution implements low-cost and green in-situ remediation, showing that the method has a long-term and in-situ application potential.

### BRIEF DESCRIPTION OF DRAWINGS

FIG. 1 shows a structure of a bioelectrode for increasing an abundance of cable bacteria and a state diagram of being inserted into sediments according to an embodiment.

FIG. 2 shows an abundance of cable bacteria added in sediments of a bioanode started at a −200 mV to 400 mV constant potential according to Embodiments 1-4, and comparison with Comparative Example 1 and Comparative Example 2.

FIG. 3(A) shows cable bacteria on a surface of a bioanode started at a 0 mV constant potential according to Embodiment 1.

FIG. 3(B) shows cable bacteria added in sediments of a bioanode started at a Om V constant potential according to Embodiment 1.

FIG. 4 respectively shows abundances of cable bacteria added in sediments of a bioanode started at a 0 mV constant potential according to Embodiment 1 and a bioanode started at a 1000Ω resistance according to Embodiment 5. A potential stabilized by the self-started bioanode is about 50 mV, and oxygen content of the overlying water is lower than 0.4 mg/L.

### DETAILED DESCRIPTION OF EMBODIMENTS

To illustrate the technical solutions of the present invention more clearly, the following examples are listed. Raw materials, reactions and post-treatment means presented in the embodiments, unless otherwise stated, are common raw materials on the market and technical means well known to those skilled in the art.

The cable bacteria used in the present invention can be from Candidatus Electrothrix applied to freshwater sediment remediation and Candidatus Electronema applied to seawater sediment remediation. The Candidatus Electrothrix can be enriched from Wenta Park River Section (22°45′N, 113°16′E), Ronggui Town, Shunde District, Foshan, Guangdong, and the Candidatus Electronema can be enriched from Xiwan Mangrove Park (22°35′N, 113°50′E), Baoan District, Shenzhen, Guangdong.

A method for testing an abundance of cable bacteria in the present invention is as follows: using a PowerSoil kit to extract DNA in surface sediments, and using 16s RNA sequencing to obtain the abundance of the cable bacteria.

FIG. 1 shows a structure of a bioelectrode for increasing an abundance of cable bacteria and a state diagram of being inserted into sediments according to an embodiment.

### Embodiment 1

A bioelectrode for increasing an abundance of cable bacteria includes a carbon brush anode (having a diameter of 1 cm and a length of 5 cm), a carbon brush cathode (having a diameter of 1 cm and a length of 8 cm), and a partition (used to form an anode chamber and fasten the entire apparatus in the sediments, with a thickness of 5 mm) made of a PVC material;

The anode is fastened to a base and horizontally disposed, and the cathode is fastened to a fastening bar, and disposed perpendicular to the anode;

The anode and the cathode are separated by using the partition, and a potentiostat is connected between the anode and the cathode.

A using method of a bioelectrode for increasing an abundance of cable bacteria includes the following steps:
S1: Select 30 mL of sediments containing cable bacteria that have been detected in a laboratory, add 0.05 mol/kg of Na₂S and mix well to obtain a mixture, put the mixture in a 50 mL beaker, and then put the mixture in an oxygen-rich (oxygen content being about 6 mg/L) water body for 21 d, until sediments containing the cable bacteria in a relatively ideal state are obtained;
S2: Introduce 5 g of the above-mentioned sediments into 500 g of to-be-remediated sediments in a 1 L beaker, and mix evenly to realize inoculation of cable bacteria in the to-be-remediated sediments;
S3: Add enough overlying water until a depth of 8 cm, to form a system, and then adopt a silent pump to keep aeration (maintain oxygen content to 6 mg/L) lasting for 21 d;
S4: Insert the bioelectrode for increasing the abundance of the cable bacteria into the sediments, make the anode be positioned on a surface of the sediments, and the cathode be basically positioned on a surface of the overlying water, maintain a constant potential of the anode (set to 0 mV) with a potentiometer, stop the aeration, and insert a reference electrode (Ag/AgCl, 3M KCl) in the sediments for controlling the constant potential of the anode;
S5: Inside of the anode gradually reaches an anaerobic condition (oxygen content is lower than 0.4 mg/L), maintain this state for 30 d, test the abundance of the cable bacteria in the anode, and record the abundance as D.

### Embodiments 2 to 4

Physical parameters and using methods of Embodiments 2 to 4 are the same as those of Embodiment 1, and the only difference is that the potentiometer maintains the constant potential of the anode as shown in Table 1 below.

TABLE 1
Constant Potential Values of the Anode Maintained
by the Potentiometer in Embodiments 2 to 4

| Embodiment | Constant Potential Value (mV) |
|---|---|
| Embodiment 2 | −200 |
| Embodiment 3 | 200 |
| Embodiment 4 | 400 |

### Embodiment 5

Embodiment 5 is set up to make the apparatus implement in-situ remediation application without energy supply and expand the application range of the apparatus. The physical parameters and usage method of Embodiment 5 are the same as those of Embodiment 1. The only difference is that in Embodiment 5, a 1000Ω resistor is used to replace the potentiostat, so that the apparatus can be self-started without power and energy, and the potential of the successfully started anode is about 50 mV.

### Comparative Example 1

A preparation method for the system in Comparative Example 1 is the same as that in Embodiment 1, and no bioelectrode of any type is inserted during use.

### Comparative Example 2

A preparation method for the system in Comparative Example 2 is the same as that in Embodiment 1, and the only difference is that the potentiometer maintains the constant potential of the anode to 550 mV.

### Test Example

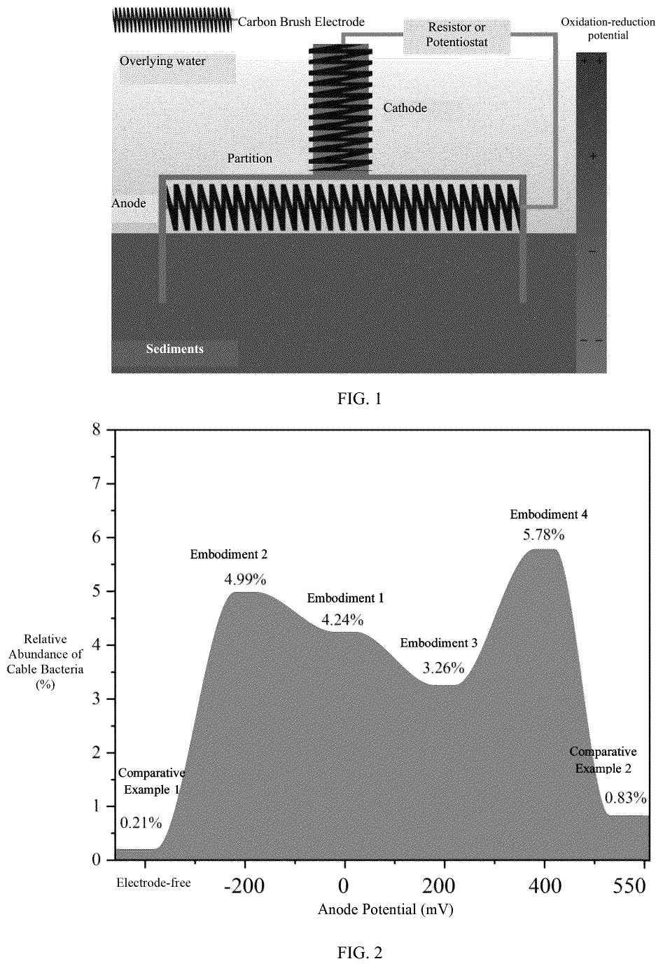

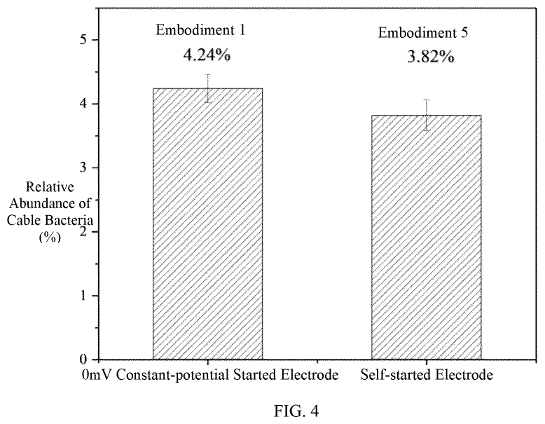

A relative abundance of the cable bacteria is tested for Embodiments 1 to 5 and Comparative Examples 1 to 2 respectively, and the obtained results D are compared, as shown in Table 2, FIG. 2 and FIG. 4.

TABLE 2
D Values of Embodiments and Comparative Examples

| Sample | D (%) |
|---|---|
| Embodiment 1 | 4.24 |
| Embodiment 2 | 4.99 |
| Embodiment 3 | 3.26 |
| Embodiment 4 | 5.78 |
| Embodiment 5 | 3.82 |
| Comparative Example 1 | 0.21 |
| Comparative Example 2 | 0.83 |

It can be learned from Table 2, that the D value obtained by the technical means of Embodiments 1 to 4 fluctuates in a range of 3.26-5.78%, indicating that the abundance of the cable bacteria has a relatively ideal increase. By contrast, the abundance of the cable bacteria in Comparative Example is only 0.21%, indicating that the bioelectrode of the present invention is advanced. The abundance of the cable bacteria in Embodiment 5 can reach 3.82%, indicating that the bioelectrode of the present invention has the potential of in-situ remediation without energy consumption in the field.

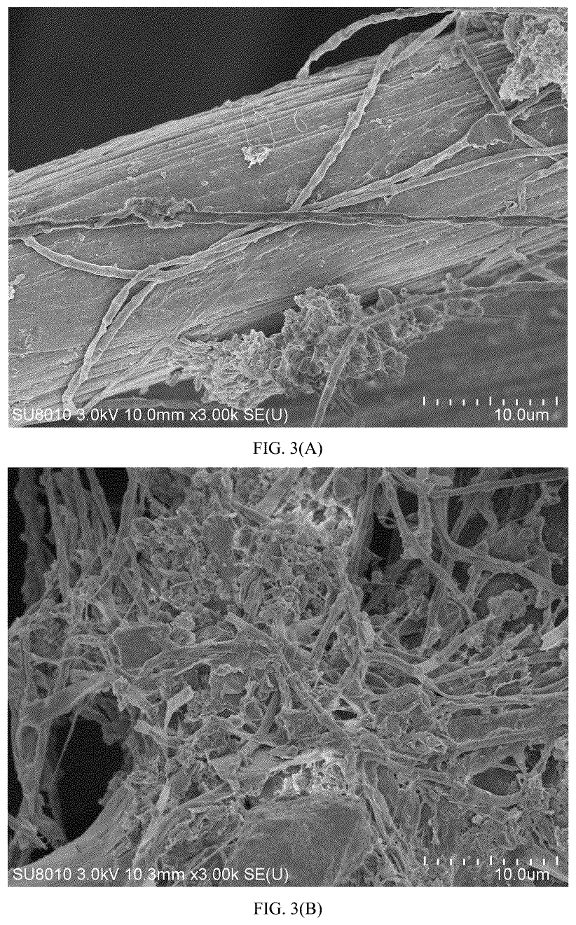

To further confirm the above conclusion, we conduct an SEM scanning electron microscopy test on the electrode in Embodiment 1 and the sediments, and the results are shown in FIG. 3(A) to FIG. 3(B) respectively. It can be learned that there are a large number of cable bacteria attached to a relevant structure in an electron microscopy image.

FIG. 4 respectively shows abundances of cable bacteria added in sediments of a bioanode started at a 0 mV constant potential according to Embodiment 1 and a self-started bioanode according to Embodiment 5. A potential stabilized by the self-started bioanode is about 50 mV, and oxygen content of the overlying water is lower than 0.4 mg/L.

It is apparent to those skilled in the art that the present invention is not limited to the details of the above-described exemplary embodiments, and the present invention can be embodied in other specific forms without departing from the spirit or essential characteristics of the present invention. Therefore, the present embodiments are to be considered as illustrative and not restrictive, and the scope of the present invention is defined by the appended claims instead. All changes in the meaning and scope of equivalent elements are included in the present invention.

In addition, it should be understood that although this specification is described in terms of embodiments, not every embodiment includes only an independent technical solution. This description of the specification is for clarity only, and those skilled in the art should take the specification as a whole. The technical solutions in the embodiments can also be appropriately combined to form other implementations that can be understood by those skilled in the art.

1. A bioelectrode for increasing an abundance of cable bacteria, wherein the bioelectrode comprises an anode, a cathode, and a partition, wherein;
the anode is horizontally disposed, and the cathode is disposed perpendicular to the anode;
and the anode and the cathode are separated by using the partition.

2. The bioelectrode for increasing an abundance of cable bacteria according to claim 1, wherein the anode and/or the cathode are/is made of a carbonaceous material.

3. The bioelectrode for increasing an abundance of cable bacteria according to claim 1, wherein the partition is made of an insulation material.

4. The bioelectrode for increasing an abundance of cable bacteria according to claim 1, wherein the anode and/or the cathode are/is carbon brush electrodes/a carbon brush electrode.

5. The bioelectrode for increasing an abundance of cable bacteria according to claim 1, wherein a resistor or a potentiostat is connected between the anode and the cathode.

6. A using method of the bioelectrode for increasing an abundance of cable bacteria according to claim 1, comprising the following steps:
S1: inoculating cable bacteria into to-be-remediated sediments;
S2: keeping aeration lasting for 14 to 21 d in overlying water added in step S1 or original overlying water;
S3: inserting the bioelectrode for increasing the abundance of the cable bacteria into the sediments, keeping a constant potential of the anode, stopping the aeration, and sealing the entire system;
S4: maintaining for 5 to 60 d after the system reaches an anaerobic condition, taking the sediments again to test the abundance of cable bacteria in the sediments, and recording the abundance of the cable bacteria as D.

7. The using method for the bioelectrode for increasing an abundance of cable bacteria according to claim 6, wherein the anaerobic condition is that the oxygen content in the overlying water is less than or equal to 0.4 mg/L.

8. The using method for the bioelectrode for increasing an abundance of cable bacteria according to claim 6, wherein the constant potential of the anode is −200 mV to 400 mV.

9. The using method for the bioelectrode for increasing an abundance of cable bacteria according to claim 6, wherein the method for testing the abundance of the cable bacteria in the sediments is a 16s RNA sequencing method.
